(19) United States
(12) Patent Application Publication
(10) Pub. No.: US 2022/0004167 A1
(43) Pub. Date: Jan. 6, 2022

(54) EXOSUIT HISTORICAL DATA

(71) Applicant: X Development LLC, Mountain View, CA (US)

(72) Inventors: Kathryn Jane Zealand, Palo Alto, CA (US); Joseph Hollis Sargent, San Francisco, CA (US); Georgios Evangelopoulos, Venice, CA (US); Elliott J. Rouse, Ann Arbor, MI (US)

(21) Appl. No.: 17/367,281

(22) Filed: Jul. 2, 2021

(30) Foreign Application Priority Data
Jul. 2, 2020 (GR) 20200100393

(51) Int. Cl. G05B 19/4155; G06N 20/00; G06F 16/23

(52) U.S. Cl. CPC G05B 19/4155; G05B 2219/40305; G06F 16/2379; G06N 20/00

(57) ABSTRACT

Methods, systems, and apparatus, including computer programs encoded on computer storage media, for generating, using, or both, exosuit historical data. In some implementations, (i) sensor data generated by sensors of an exosuit worn by a user and (ii) control data indicating actions performed by or control signals generated by the exosuit based on the sensor data while worn by the user are received. The sensor data and the control data are added to a database that includes historical data describing use of the exosuit over time by the user. A control scheme of the exosuit is customized for the user by updating the one or more machine learning models or settings that govern the application of the one or more machine learning models. Forces provided by one or more actuators of the exosuit are controlled using the updated one or more machine learning models or the updated settings.

### CROSS-REFERENCE TO RELATED APPLICATION

This application claims priority to Greek Application No. 20200100393, filed Jul. 2, 2020, the contents of which are incorporated by reference in its entirety herein.

### FIELD

This disclosure generally relates to exosuits, such as exoskeletons.

### BACKGROUND

A person may wear an exosuit. The exosuit can include a number of sensors, such as gyroscopes and accelerometers. The exosuit can include control mechanisms that control the movement of the device. Some control mechanisms can include actuators and end effectors.

The exosuit can be any appropriate size. For instance, an exosuit can cover only an upper portion of a user's body. In some examples, an exosuit can include sensors and control mechanisms in only a portion of the exosuit. For example, an exosuit can cover an upper portion of a user's body, e.g., be a shirt, while only including sensors, control mechanisms, and the like in the left arm of the exosuit.

### SUMMARY

A system can generate an exosuit profile history using sensor data, control data, or both, from an exosuit. Sensor data can indicate information about an environment in which the exosuit was used, such as detected user movement. The control data can indicate actions or control values performed by the exosuit in response to the detected sensor data.

The system can use the exosuit profile history to track an effectiveness of machine learning models that generate the control signals, actuator control, or both. This can enable the exosuit to more accurately determine an action to perform when the exosuit receives similar input data in the future.

For instance, when the exosuit has difficulty distinguishing when the sensor data indicates that the user is running or walking up stairs, analysis of the data in the exosuit profile history can improve the exosuit's response in similar future situations. The exosuit can train a model specific to the user, or a group to which the user belongs, to improve the exosuit's accuracy.

In some implementations, the system can use the exosuit profile history to track progress of injury recovery, determine injury status, determine user responsiveness to exosuit control, or a combination of two or more of these. The system, or the exosuit, can use the tracked information to provide personalized data to a user. For instance, the system can provide personalized metrics about the progress of injury recovery. The system can provide a recommendation of how the user can better respond to or interact with the exosuit.

In general, one aspect of the subject matter described in this specification can be embodied in methods that include the actions of receiving, sensor data generated by one or more sensors of an exosuit that indicates movement of the exosuit while the exosuit is worn by a user; adding the sensor data to a database of historical data describing assistance provided to the user by the exosuit; analyzing the historical data including the sensor data to determine one or more personalized metrics for the user that are indicative of a level of usage of the exosuit; and providing the one or more personalized metrics for presentation to the user. Other embodiments of this aspect include corresponding computer systems, apparatus, computer program products, and computer programs recorded on one or more computer storage devices, each configured to perform the actions of the methods. A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

In one general aspect, a method includes: receiving, by one or more computing devices, (i) sensor data generated by sensors of an exosuit worn by a user and (ii) control data indicating actions performed by or control signals generated by the exosuit based on the sensor data while worn by the user, where the control data is determined using one or more machine learning models; adding, by the one or more computing devices, the sensor data and the control data to a database includes historical data describing use of the exosuit over time by the user; customizing, by the one or more computing devices, a control scheme of the exosuit for the user by updating the one or more machine learning models or settings that govern application of the one or more machine learning models, where the control scheme is customized using the historical data for the user; and controlling, by the one or more computing devices, forces provided by one or more actuators of the exosuit using the updated one or more machine learning models or the updated settings.

Implementations may include one or more of the following features. For example, in some implementations, the method includes receiving data that identifies user input that indicates whether an action by the exosuit should have been performed; and adding the data that identifies the user input to the database, where updating the one or more machine learning model includes updating the one or more machine learning models using the historical data and the user input.

In some implementations, the one or more machine learning models includes a generic model; and updating the one or more machine learning models for the user using the historical data includes updating the generic model using a transfer learning process and the sensor data received by the exosuit during use of the exosuit by the user.

In some implementations, updating the one or more machine learning models using the historical data includes updating the one or more machine learning models using an unsupervised learning process and movement patterns of the user identified from the historical data.

In some implementations, updating the one or more machine learning models using the historical data includes updating the one or more machine learning models using a reinforcement learning process and data that identifies user input received from a user who wore the exosuit, the data indicating an assistance measure for the exosuit.

In some implementations, the database includes second sensor data captured by sensors included in a plurality of other exosuits respectively worn by other users and second control data indicating actions performed by or control signals respectively generated by the other exosuits while worn by the corresponding other users; and updating the one or more machine learning models includes updating the one or more machine learning models using the sensor data, the control data, the second sensor data and the second control data that are included in the database of the historical data to obtain the updated one or more machine learning models.

In some implementations, the method includes: determining, using some of the sensor data or some of the control data from the database, an exosuit activity type; and selecting, using the exosuit activity type, a particular machine learning model from a plurality of machine learning models for use by the exosuit when the user is wearing the exosuit.

In some implementations, the exosuit activity type includes one of: lifting an object, walking, sitting, standing, running, walking up stairs, or writing.

In another general aspect, a method includes: receiving sensor data generated by one or more sensors of an exosuit that indicates movement of the exosuit while the exosuit is worn by a user; adding the sensor data to a database of historical data describing assistance provided to the user by the exosuit; analyzing the historical data including the sensor data to determine one or more personalized metrics for the user that are indicative of a level of usage of the exosuit; and providing the one or more personalized metrics for presentation to the user.

Implementations may include one or more of the following features. For example, in some implementations, the one or more personalized metrics indicate at least one of: a difference in an amount of assistance that the exosuit provided during a first time period compared to a second amount of assistance the exosuit provided during a second period of time prior to the first time period; an indication of whether the user properly used the exosuit; a difference in an amount of activity by the user during a third period of time compared to a fourth period of time prior to the third period of time; or an assistance index determined based on at least one of a level of usage of the exosuit or a level of benefit from using the exosuit.

In some implementations, providing the personalized metrics includes displaying the personalized metrics on a display included in the exosuit.

In some implementations, providing the personalized metrics includes providing the personalized metrics to an external device to cause the external device to present the personalized metrics.

In some implementations, the external device includes one of a mobile phone or a smart watch.

In some implementations, receiving the sensor data includes receiving the sensor data at a server system over a communication network; and providing the one or more personalized metrics includes providing, by the server system, the one or more personalized metrics over the communication network to a device associated with the user.

In some implementations, the exosuit includes a powered exoskeleton, mechanized clothing, or mechanically assistive clothing.

In some implementations, the method includes receiving data indicating actions performed by the exosuit or control signals generated by the exosuit, where the one or more personalized metrics are further based on the actions performed by the exosuit or control signals generated by the exosuit.

In some implementations, the method includes providing, for presentation to the user, data indicating a recommendation for changing a manner of using the exosuit, where the recommendation is determined based on the historical data.

In some implementations, the method includes: receiving first sensor data for a first period of time; storing the first sensor data in the database of historical data; receiving second sensor data for a second period of time that is a different time period than the first period of time; and storing the second sensor data in the database of historical data.

In general, another aspect of the subject matter described in this specification can be embodied in methods that include the actions of receiving (i) sensor data generated by sensors of an exosuit worn by a user and (ii) control data indicating actions performed by or control signals generated by the exosuit based on the sensor data and while worn by the user, wherein the control data is determined using a machine learning model; adding the sensor data and the control data to a database comprising historical data describing use of the exosuit over time by the user; updating the machine learning model using the historical data to obtain an updated machine learning model; and providing the updated machine learning model to the exosuit for controlling actions of the exosuit. Other embodiments of this aspect include corresponding computer systems, apparatus, computer program products, and computer programs recorded on one or more computer storage devices, each configured to perform the actions of the methods. A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

The foregoing and other embodiments can each optionally include one or more of the following features, alone or in combination. The one or more personalized metrics can indicate at least one of: a difference in an amount of assistance that the exosuit provided during a first time period compared to a second amount of assistance the exosuit provided during a second period of time prior to the first time period; an indication of whether the user properly used the exosuit; a difference in an amount of activity by the user during a third period of time compared to a fourth period of time prior to the third period of time; or an assistance index determined based on at least one of a level of usage of the exosuit or a level of benefit from using the exosuit. Providing the personalized metrics can include displaying the personalized metrics on a display included in the exosuit.

In some implementations, providing the personalized metrics can include providing the personalized metrics to an external device to cause the external device to present the personalized metrics. The external device can be one of a mobile phone or a smart watch. Receiving the sensor data can include receiving the sensor data at a server system over a communication network. Providing the one or more personalized metrics can include providing, by the server system, the one or more personalized metrics over the communication network to a device associated with the user.

In some implementations, the exosuit can include a powered exoskeleton, mechanized clothing, or mechanically assistive clothing. The method can include receiving data indicating actions performed by the exosuit or control signals generated by the exosuit. The one or more personalized metrics can be based on the actions performed by the exosuit or control signals generated by the exosuit. The method can include providing, for presentation to the user, data indicating a recommendation for changing a manner of using the exosuit. The recommendation can be determined based on the historical data.

In some implementations, the method can include receiving first sensor data for a first period of time; storing the first sensor data in the database of historical data; receiving second sensor data for a second period of time that is a different time period than the first period of time; and storing the second sensor data in the database of historical data. The method can include receiving data that identifies user input that indicates whether an action by the exosuit should have been performed; and adding the data that identifies the user input to the database. Updating the machine learning model can include updating the machine learning model using the historical data and the user input.

In some implementations, the machine learning model can be a generic model. Updating the machine learning model for a user using the historical data can include updating the generic model using a transfer learning process and the sensor data received by the exosuit during use of the exosuit by the user. Updating the machine learning model using the historical data can include updating the machine learning model using an unsupervised learning process and user movement patterns identified from the historical data. Updating the machine learning model using the historical data can include updating the machine learning model using a reinforcement learning process and data that identifies user input received from a user who wore the exosuit. The data can indicate an assistance measure for the exosuit.

In some implementations, the database can include second sensor data captured by sensors included in a plurality of other exosuits that are respectively worn by other users and second control data indicating actions performed by or control signals generated by the respective other exosuits while worn by the corresponding users. Updating the machine learning model can include updating the machine learning model using the sensor data, the control data, the second sensor data and the second control data that are included in the database of historical data to obtain the updated machine learning model. The method can include determining, using some of the sensor data or some of the control data from the database, an exosuit activity type; selecting, using the exosuit activity type, a particular machine learning model from a plurality of machine learning models for use by the exosuit when the user is wearing the exosuit. The exosuit activity type can include one of: lifting an object, walking, sitting, standing, running, walking up stairs, or writing.

The subject matter described in this specification can be implemented in various embodiments and may result in one or more of the following advantages. In some implementations, training a machine learning model using exosuit historical data can cause an exosuit that uses the machine learning model to more accurately select an action to perform based on received sensor data, enable an exosuit to actuate without determining a specific action to perform, or both. For instance, when a user performs a new or otherwise unspecified activity, an activity that is an edge case or a transition, or both, the exosuit can actuate using the machine learning model without selecting a specific action to perform. In some implementations, presentation of personalized assistance metrics generated using exosuit historical data can improve exosuit interaction with a user, efficiency, or both.

In some implementations, presentation of personalized assistance metrics generated using exosuit historical data can improve exosuit user safety, exosuit user rehabilitation, exosuit user athletic performance, or a combination of two or more of these. For example, a system can use the historical data to determine recommended safety changes for an exosuit user. When the historical data indicates that the exosuit user walks a particular way, e.g., when the center of pressure for the user's foot is close to the edge of the foot, a machine learning model can analyze the historical data and determine a recommendation for a safer way for the exosuit user to walk, e.g., placing pressure closer to the center of the foot. This can improve the exosuit user's safety, e.g., by presenting a recommendation that would enable the exosuit user to be more stable when walking.

In some implementations, a system can determine metrics that describe diagnostic benefits of the exosuit, a way in which an exosuit user can improve a medical condition, e.g., a knee injury, or both. For instance, the system can analyze the historical data to determine a way to use the exosuit, or perform another activity, that can enable an injury to heal more quickly, e.g., determine a recommendation on a different way to walk so that a knee injury can heal more quickly.

In some implementations, a system can determine metrics that can enable athletic performance improvements. For instance, the system can determine, using the historical data, a recommendation about how an exosuit user can run more efficiently, run more quickly, or both. A system can present the recommendation to the exosuit user.

The details of one or more implementations of the subject matter described in this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 depicts an example of an environment that includes an exosuit and a training system.

FIG. 2 depicts an example of an environment in which a device receives, for presentation, personalized metrics or a recommendation from a personalized metrics system.

FIG. 3 is a flow chart illustrating an example process for controlling an exosuit using sensor data collected over time.

Like reference numbers and designations in the various drawings indicate like elements.

### DETAILED DESCRIPTION

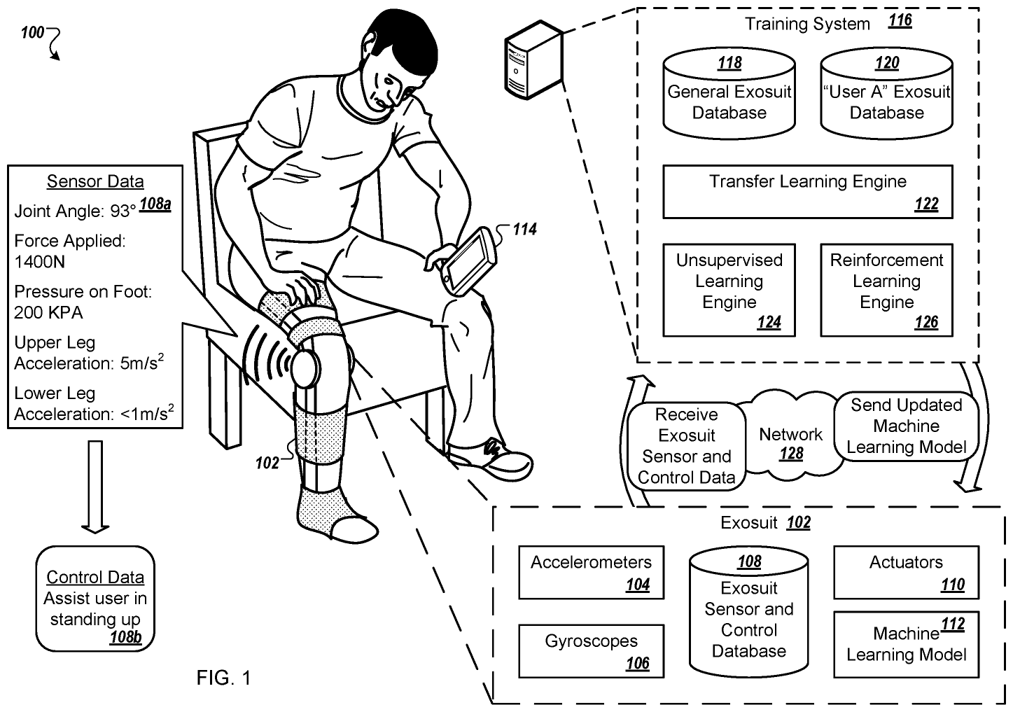

FIG. 1 depicts an example of a system 100 that includes an exosuit 102 and a training system 116. The exosuit 102 can assist a user in performing one or more activities, such as walking, running, going up stairs, or lifting an object. For instance, the exosuit 102 can be part of a leg brace to assist a person who was in an accident, or otherwise has difficulty walking, with walking.

The exosuit 102 uses a machine learning model 112 to apply actuation based on contextual information about the environment in which the exosuit 102 is located. The contextual information can include data received from sensors included in the exosuit 102, among other sources, e.g., data about a user of the exosuit. For instance, when the exosuit 102, e.g., while worn by a user, is at the bottom of a staircase, the exosuit 102 can determine to assist a user in walking up the stairs, at a measured pace, rather than determining to assist a user in running. In some implementations, multiple machine learning models 112 are used, with different models used for different types of activities, e.g., with one model 112 being used for walking, another model 112 used for a sitting movement, another for a standing movement, another model 112 for ascending stairs, etc. The exosuit 102 may dynamically select between different models 112 depending on the context and sensor data to generate the correct instructions to the actuators of the exosuit 102.

The machine learning models 112 can be, for example, neural networks, classifiers, support vector machines, regression models, reinforcement learning models, clustering models, decision trees, random forest models, genetic algorithms, Bayesian models, or Gaussian mixture models. Different types of models can be used together as an ensemble or for making different types of predictions. Other types of models can be used, even if they are not of the machine learning type. For example, statistical models and rule-based models can be used in some implementations. Training of the models 112 can incrementally or iteratively update the values of parameters in the models 112. In the case of neural networks, backpropagation can be used to alter neural network weights for neurons at various layers of the neural network. As discussed below, other techniques including clustering, unsupervised training, and reinforcement learning can also be used to train the models 112.

In some implementations, the exosuit 102 can use the machine learning model 112 to determine the action to perform. In some implementations, the exosuit 102 can use the machine learning model 112 to perform an action without determining a specific action to perform. For instance, the machine learning model 112 can output data for an actuator profile that the exosuit uses to perform an action that is not necessarily related to a known action, e.g., move forward, backward, or turn.

Similarities in contextual information for different actions, e.g., how a user moves their legs when running and walking up stairs, can cause the exosuit 102 to incorrectly select an action to perform. For instance, the exosuit 102 might determine to help a user walk up a staircase when the exosuit 102 should have determined to assist a user in running.

The training system 116 receives, from the exosuit 102, sensor data, control data, or both, that were generated by the exosuit 102 during use, e.g., when the exosuit 102 incorrectly or correctly determined an action to perform, such as to help a user walk up a staircase. The training system 116 uses the received data to update the machine learning model 112, e.g., to improve an accuracy of the machine learning model 112 in predicting an activity or applying actuation. This in turn enables the exosuit 102 to use an updated machine learning model 112 to more accurately apply actuation, or predict an activity to perform based on contextual information for the exosuit 102 in the future.

The exosuit 102 can be any appropriate type of mechanized assistance device. The exosuit 102 can be a powered exoskeleton, mechanized clothing, or mechanically assistive clothing. For example, the exosuit 102 can be part of a piece of clothing, such as a pair of pants. This can enable the exosuit 102 to be difficult to detect, e.g., visually without touching the pair of pants.

The exosuit 102 can include multiple sensors. Some examples of sensors include accelerometers 104 and gyroscopes 106. Other sensors include force sensors, position sensors, odometry sensors, encoders for position or rotation, tilt sensors, contact sensors, and so on. While a user is using the exosuit 102, the sensors generate data 108a about the exosuit's 102 use. For instance, the sensors generate data 108a that indicates a movement direction and movement speed of the exosuit 102. The sensors can generate data 108a that identifies rotational parameters for the exosuit 102, such as whether the exosuit 102 was rotated, e.g., when a user turned or during normal movement. The sensors can detect a variety of conditions, including those caused by a user, e.g., resulting from user movement or input, as well as those caused by the exosuit 102, e.g., resulting from movements of motors or other actuators of the exosuit 102.

The exosuit 102 can store the data 108a generated by the sensors in a database 108. For instance, as the sensors generate data 108a, the exosuit 102 can receive the data 108a and store the data 108a in the database 108. The database 108 can include sensor data collected over time, e.g., during exosuit use by a user. The exosuit 102 can store data in the database 108 for a predetermined period of time, e.g., until the exosuit 102 sends the data from the database 108 to the training system 116.

The exosuit 102 can use the data 108a to control exosuit action, provide feedback to a user, or both. For example, the exosuit 102 can include multiple controllers, e.g., actuators 110. The exosuit 102 can analyze the data 108a to determine an action the user is trying to perform and send control data 108b to a controller that identifies that action or a corresponding action for the controller to perform. For instance, when the exosuit 102 determines that a user is trying to stand up, the exosuit can send the control data 108b to an actuator 110 that indicates that the actuator 110 should rotate to assist the user in standing up. The actions of the exosuit 102 can include exosuit movement, applying force to a particular area, e.g., part of a user, or both.

The exosuit 102 can store data in the database 108 that indicates an action the exosuit 102 performed based on sensor data. For instance, in the above example, the exosuit 102 can store data that indicates that the exosuit 102 assisted the user in standing up. Data in the database 108 can associate exosuit actions with sensor data. For instance, the exosuit 102 can create an entry in the database that indicates that when the joint angle was 93°, the force applied was 1400N, the foot pressure was 200 KPA, the upper leg acceleration was 5 m/s² and the lower leg acceleration was less than 1 m/s², the exosuit 102 assisted the user in standing up.

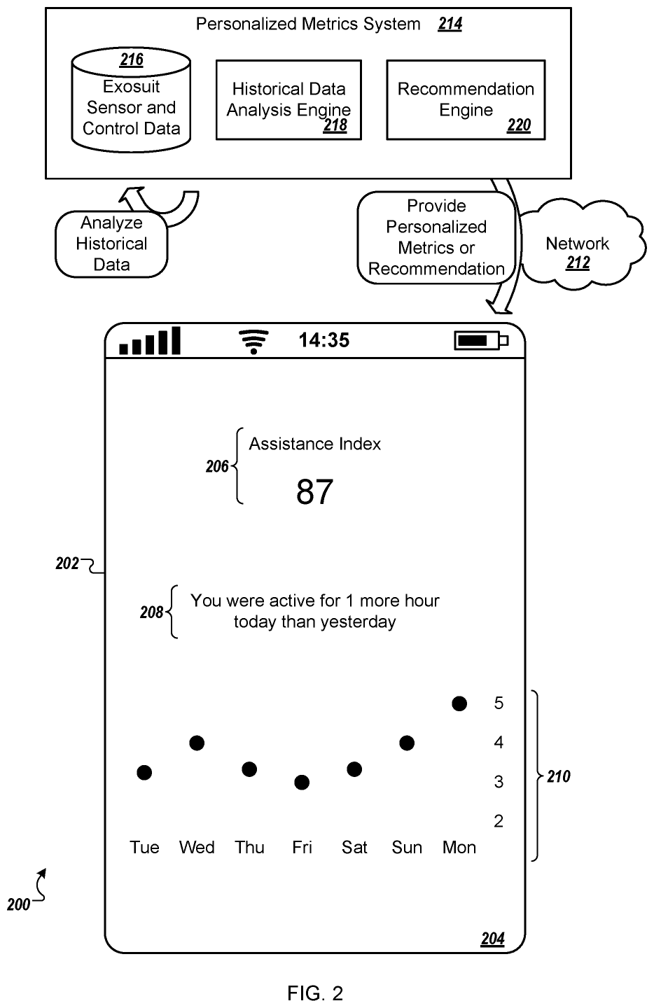

In some implementations, the exosuit 102 can include a feedback device, e.g., a display or a communication device that provides feedback to another device such as a user device 114, as described in more detail below with reference to FIG. 2. The feedback device can provide a user with information about the exosuit 102, such as information about an assisted arm movement.

The exosuit 102 includes the machine learning model 112 used in the control of the exosuit 102. For example, the exosuit 102 uses the machine learning model 112 to determine when and how to assist a user in standing up. The machine learning model 112 receives, as input, data from various sensors in the exosuit 102. The machine learning model 112 uses the received input to predict whether to assist a user and in what manner to assist the user, e.g., to determine which actuators to activate, the speed and position for movement, an amount of force to apply, and so on. For instance, the machine learning model 112 can implicitly account for or determine whether the user is trying to stand up and requires assistance from the exosuit 102 or does not require assistance, e.g., and the user is merely shifting their leg.

The exosuit 102 can include any appropriate machine learning model 112. In some implementations, the exosuit 102 is initialized with a generic machine learning model 112, e.g., that was trained using data for multiple different people. The generic machine learning model 112 can be a classifier that predicts actions. The generic machine learning model 112 can be a regressor that outputs physical quantities, e.g., current, for control of an exosuit. In some implementations, the exosuit 102 is initialized with a machine learning model 112 specific to a user, e.g., that was previously trained for the user.

The exosuit 102 can update the machine learning model 112 by sending data for the exosuit 102 to a training system 116. The data for the exosuit can include sensor data, control data, or both.

The training system 116 receives the data for the exosuit and uses the data to train a machine learning model. For instance, upon receipt of data for a user A from the exosuit 102, the training system 116 can store the data in a general exosuit database 118, a user A exosuit database 120, or both. The training system 116 can then use data from the database 118 or the database 120, or both, to train a machine learning model. Training the machine learning module to personalize controller tuning to a particular person who has used the exosuit 102, e.g., user A, can improve the accuracy of the machine learning model 112 when predicting actions the user is taking, actions to perform, or both, for when the particular person uses an exosuit again in the future.

The training system 116 can include a copy of the machine learning model 112 or otherwise have access to the machine learning model 112. For instance, when the training system 116 is implemented on one or more servers, the training system 116 can receive, with the sensor data, the control data, or both, an identifier for the exosuit 102. The training system 116 can use the identifier to determine one or more machine learning models associated with the exosuit 102 that should be updated using the received data. When the training system 116 is implemented on the exosuit 102, the training system 116 can access the copy of the machine learning model 112 stored in a memory of the exosuit 102. When the training system 116 is implemented on the user device 114, the training system 116 can access a local copy of the machine learning model 112, or request a copy of the machine learning model 112 from the exosuit 102.

The training system 116 can include a transfer learning engine 122. The transfer learning engine 122 can use the received data to train a generic machine learning model. The training can customize the generic machine learning model for a specific user associated with the received data, e.g., the user A. For example, the training system 116 can receive sensor data, control data, or both, from one or more physical training sessions in which the user A used the exosuit 102 to perform one or more actions. The physical training sessions can be part of a physical therapy process or any other appropriate type of training. The transfer learning engine 122 can use this data as input to a training process to customize a generic machine learning model that was originally created using data for multiple users stored in the general exosuit database 118. This process can adjust the generic machine learning model to the specific user and increase the accuracy of the exosuit 102 when performing actions based on sensor data generated from the specific user's movements.

The sensor data used during the training process, e.g., by any of the learning engines, can indicate the conditions present for the exosuit during a particular period of time. The controller data can indicate the inputs to the machine learning model 112 for the particular period of time. The sensor data can also include subsequent sensor data, detected after the particular period of time, that identifies the actual user movement.

The training system 116 can use any appropriate type of data during the training process, whether received from a sensor or another device. For instance, the training system 116 can use data that indicates actual movement of the exosuit, the user, or both, given a context in which the exosuit is used, user input, or both. The user input can be input received before or after the exosuit 102 applies actuation for an activity and can indicate the activity during which the exosuit 102 applied actuation. In some examples, the training system 116 can receive data from an activity classification model, e.g., running on the training system 116, that analyzes data to predict an activity during which the exosuit 102 applied actuation.

In some implementations, the training system 116 can interpret the received data to infer what the appropriate control data would have been to support the movement of the user. The training system 116 can determine the appropriate control data using another model, e.g., another machine learning model. The other model can be a larger, more complex model, have a different architecture than the machine learning model, or both. The training system 116 can provide, as input to the other model, data that indicates an activity performed by the exosuit 102, e.g., the activity predicted by the exosuit, an activity label for an action a person performed when the exosuit 102 performed the activity, e.g., the activity intended by the user, or both. The training system 116 can receive, as output from the other model, corrected control data. The activity label can be generated using feedback received from the person wearing the exosuit 102, e.g., the activity label can be a user defined label. A user device can receive the feedback from the user, e.g., when the user presses a button on a screen of the user device, provides an audible command, or both.

In some implementations, the training system 116 can provide, to the other model, sensor data as input. The sensor data can be electromyography (“EMG”) data, data from pressure sensors, data from force sensors, or a combination of two or more of these.

The training system 116 can use the correct control data to further train the machine learning model 112 for a user's own behavior and idiosyncratic movement style. For instance, the transfer learning engine 122 can use the correct control data as input to adapt or refine the machine learning model 112 for each user based on their actual movement patterns tracked over time. Use of the correct control data as input can enable the transfer learning engine to be unsupervised, e.g., not require user feedback on the exosuit's 102 actions such that the user feedback would be used during the training process.

The training system 116 can include an unsupervised learning engine 124. The unsupervised learning engine 124 can process data from the general exosuit database 118 to create one or more machine learning models. For example, the unsupervised learning engine 124 can analyze data from the general exosuit database 118 to determine datasets with sensor data based common traits, such as common walking patterns, common movements, common physical conditions, common activities performed, common age groups, or other lifestyle commonalities. Some examples of common physical conditions can include having a broken leg or a broken arm. Some common activities include hiking, running, and biking.

The unsupervised learning engine 124 can use the data in a dataset for a common trait, e.g., having a particular walking pattern, to generate a machine learning model for that common trait. The unsupervised learning engine 124 can use the data in the dataset as input to the training process. The unsupervised learning engine 124 can use unlabeled data for the training process. The unsupervised learning engine 124 does not need data that identifies user feedback based on an exosuit's actions.

Once the machine learning model for that common trait is trained, the training system 116 can store the machine learning model in a model database of machine learning models. The model database can include labels that identify the common traits for the respective machine learning models. This can enable the training system 116 to train many models for different types of users, different types of activities, or both. For instance, the model database can include a first model for runners between the ages of five and twelve and a second model for runners between the ages of thirty and forty. Although the models are described here as applying to a particular age range, the model database and the training system 116 need not identify a predicted age range for a model and an exosuit for a user outside of an age range for a model can use the model when sensor data for the user satisfies a threshold number of similarities with the sensor data used to train the model.

The training system 116 can use the received data to identify a machine learning model from the model database for the exosuit 102. For instance, the model database can identify the data used to train the model. The training system 116 can compare the received data with the various datasets used by the unsupervised learning engine 124. The training system can select the dataset that is most similar to the received data and then select the machine learning model, in the model database, for the selected dataset.

The training system 116 sends the updated machine learning model to the exosuit 102. The updated model can be a model selected from a model database for the exosuit 102, e.g., based on the received data. The updated model can be a model trained specifically for the exosuit 102, e.g., by the transfer learning engine.

In some implementations, the exosuit 102 can have access to or include a copy of the machine learning model 112. For instance, when the training system 116 stores the machine learning model 112 in a database, the training system 116 can provide a copy of the machine learning model 112 to the exosuit 102. The exosuit 102 can use the machine learning model 112 to perform actions whether or not the exosuit 102 is connected to the training system 116, e.g., using the network 128. In some examples, the training system 116 can provide the exosuit 102 with a reference to the machine learning model 112. This can enable the exosuit 102 to access a model that is trained by and stored on the training system 116 without storing the machine learning model in a memory of the exosuit 102.

The training system 116 can include a reinforcement learning engine 126. The reinforcement learning engine 126 can use the data received from the exosuit 102 to train a model for a user of the exosuit 102. In these implementations, the received data includes user feedback. The feedback can indicate an assistance quality or an accuracy of the actions performed by the exosuit 102. The exosuit 102 can receive the user feedback using any appropriate input device, e.g., a smart watch, or voice input. The user feedback can indicate what assistance the exosuit performed correctly, what assistance the exosuit performed incorrectly, an action identified by the user for a certain context, e.g., a preferred action, or a combination of two or more of these. The feedback can include a binary value, e.g., correct or incorrect. The feedback can include a scaled value, e.g., between zero and one. The feedback can indicate which assistance the exosuit should have provided, e.g., a relative preference for a user. The user feedback can be received by any appropriate type of input device. For instance, the user feedback can be text data received by a touch screen or a keyboard. The user feedback can be voice data received by a microphone, e.g., included in the exosuit 102 or the user device 114.

The reinforcement learning engine 126 can use the user feedback to improve the machine learning model for the exosuit 102, e.g., to improve how the exosuit 102 responds to sensor input. The reinforcement learning engine 126 can use the user feedback to predict custom assistance strategies for the exosuit 102 to perform based on sensor input. The custom assistance strategies can be a sequence of tasks or steps, or an assistance profile for the exosuit 102 to perform when the exosuit 102 detects particular sensor input. An assistance profile can indicate actions for an exosuit to perform, e.g., for a particular person, customized control settings, use history for a particular person, characteristics of a person who will use the exosuit 102, or a combination of two or more of these. In some examples, an assistance profile can indicate a set of actuation values across time, e.g., a pre-stored actuation such as torque as a function of time.

In some implementations, the exosuit 102 can include multiple machine learning models 112. The exosuit 102 can include a coordination machine learning model that determines which of multiple activity specific models should be used to generate control data. For instance, the exosuit 102 can include a first activity specific model for assisting a user in standing, a second activity specific model for assisting a user in running, and a third activity specific model for assisting a user in walking up stairs. When the exosuit 102 uses the second activity specific model to assist a user of the exosuit 102 in running and then detects, based on sensor input, a change in an activity type, the coordination machine learning model selects another machine learning model using the sensor input. The exosuit 102 then switches control of the controllers, e.g., actuators 110, to the other machine learning model, for instance, the first activity specific model for assisting the user in standing. However, if the coordination machine learning model incorrectly selected the other machine learning model, because the user reached a set of stairs and started to walk up the stairs, the exosuit 102 can receive user feedback that indicates the incorrect assistance provided by the exosuit 102.

The user feedback can be text data, voice data, or data that represents a property of the user, e.g., data that indicates that the user's heart rate went up or applied pressure in a direction opposite to the exosuit's 102 movement. In some examples, the user feedback can be passively entered by the user. For instance, when the exosuit 102 performs an action based on a prediction that the user is still exercising but the user begins to interact with the user device 114, the training system 116 can determine that the exosuit 102 performed the incorrect action as the user likely was not exercising while interacting with the user device 114.

The reinforcement learning engine 126 can train the coordination machine learning model based on the user feedback, the correct model selection, e.g., the third activity specific model in the above example, or both. The training can improve the coordination machine learning model's activity prediction accuracy so that an exosuit, that uses the coordination machine learning model, more accurately switches between models for various activities in the future.

In some implementations, the training system 116 can use two or more of the learning engines 122, 124, 126 based on the received data. For instance, the training system 116 can select a model from the model database, and that was trained by the unsupervised learning engine 124, using the data received from the exosuit. The training system 116 can then use the transfer learning engine 122 to train the selected model using the received data. The training system can then send the trained, selected model to the exosuit 102 for future use, e.g., assisting a user.

The training system 116 can receive data from the exosuit 102 at any appropriate time. For example, the training system 116 can train the machine learning model in the wild, e.g., while a user is using the exosuit 102. The exosuit 102 can provide the sensor data, the control data, or both, to the user device 114, e.g., using short range communications over a network 128. The training system 116 can then receive the sensor data, the control data, or both, from the user device 114, e.g., using long range communications over the network 128. The training system 116 can then update the machine learning model 112 while the user continues to use the exosuit 102. Once the machine learning model 112 is updated, the training system 116 can provide the updated model to the user device 114. The exosuit 102 then receives the updated model from the user device 114.

When the training system 116 receives data from the exosuit 102, the training system 116 can determine whether to update the machine learning model 112. For instance, if an accuracy of the machine learning model satisfies a threshold accuracy, the training system 116 can determine to skip additional training of the machine learning model.

The training system 116 can train the machine learning model 112 using a subset of the data in the general exosuit database 118, a subset of the data in the user A exosuit database 120, or both. For example, the training system 116 can train a model using data received during a predetermined time period, e.g., the past day or since the machine learning model 112 was last trained. The training system 116 can train the machine learning model 112 using heuristics based on a user's movement patterns.

In some implementations, the training system 116 or the exosuit 102 can change other exosuit parameters. For instance, the exosuit 102 can determine to generally increase or decrease assistance based on the sensor data, user feedback, or both. The exosuit 102 can determine to apply offsets, scaling factors or other adjustments to control signals output by a model. In some examples, the exosuit 102 can determine to apply context-dependent rules for a user. When the training system 116 performs any of these determinations, the exosuit 102 can provide all necessary data to the training system 116. The training system 116 can then perform the appropriate analysis and send a signal to the exosuit 102 to cause the exosuit 102 to change a corresponding parameter.

In some implementations, the training system 116 can analyze part or all of the sensor data, the control data, or both, received from the exosuit 102. For instance, the training system 116 can use labels, provided by the user, additional sensors, e.g., EMG sensors, another model trained using supervised or unsupervised learning or another controller, that indicate whether the analyzed data, e.g., control data, was a good decision or bad decision performed by the machine learning model 112. The training system can update the machine learning model 112 using supervised learning and the correct labels or correct control values for the bad decisions. The supervised learning can be performed by any appropriate component of the training system 116, e.g., a supervised learning engine. The training system 116 can analyze at least some of the data to identify patterns in the data for use in a training process, e.g. using an unsupervised learning engine. The training system 116 can analyze at least some of the data to characterize the user's most commonly encountered scenarios. As part of this process, the training system 116 can count instances of different movements or situations to know how to weight different actions that the exosuit 102 can perform.

The training system 116 is an example of a system implemented as computer programs on one or more computers in one or more locations, in which the systems, components, and techniques described in this document are implemented. The user device 114 may include a personal computer, a mobile communication device, and other devices that can send and receive data over the network 128. The network 128, such as a local area network (LAN), wide area network (WAN), the Internet, or a combination thereof, connects the exosuit 102, the user device 114, and the training system 116 (in implementations when the training system 116 is not part of the exosuit 102 or the user device 114). The training system 116 may use a single server computer or multiple server computers operating in conjunction with one another, including, for example, a set of remote computers deployed as a cloud computing service.

The training system 116 can include several different functional components, including the transfer learning engine 122, the unsupervised learning engine 124, and the reinforcement learning engine 126. The various functional components of the training system 116 may be installed on one or more computers as separate functional components or as different modules of a same functional component. For example, the transfer learning engine 122, the unsupervised learning engine 124, and reinforcement learning engine 126 of the training system 116 can be implemented as computer programs installed on one or more computers in one or more locations that are coupled to each through a network. In cloud-based systems for example, these components can be implemented by individual computing nodes of a distributed computing system.

In general, the system 110 can use tracked data about a user's use of the exosuit 102 to adjust a control scheme for the exosuit 102. The user's unique movement patterns, physiology, and preferences can be used to customized the control scheme in a manner that changes how the exosuit 102 provides support to the user. This change in the control can adjust any of various different parameters that affect how the exosuit 102 responds to and assists user movement, including which models 112 are selected for controlling different movements, the amount of force of actuation provided, thresholds and other settings for initiating or terminating force by actuators, the timing of forces applied and variations in force over time (e.g., different force curves or force application patterns), updating the training of machine learning models 112, and more.

For example, one way to adjust the control scheme for a user is to perform further training of one or more machine learning models 112 that customize the model outputs for the usage patterns and gait of the user, as indicated by the examples. In some cases, the tracked usage data may show that the user tends to stand up from sitting more gradually than the model 112 attempted (e.g., over 4 seconds rather than 2 seconds), or that the user often takes a longer gait than the model 112 has predicted (e.g., 2.5 feet rather than 2 feet). The further training with the tracked data can adjust the model parameters so the outputs are better aligned to the user's typical movements and patterns, personalized for the particular user's physiology. An advantage of this approach is the ability to continually adapt and adjust the model 112 as the user uses the exosuit 102. For example, as a user's injury heals or as the user becomes more comfortable with the exosuit 102, there may be changes in user gait, timing of movements, force provided by the user, the frequency and relative proportion of different situations and movements performed (e.g., standing up, sitting down, ascending stairs, descending stairs, walking, running, etc.). The system 100 can adjust the model 112 on an ongoing basis to tailor support to the user's needs.

In some cases, the system 100 starts with a base model 112 and further trains it for the user, based on the sensor data of the user's actual usage patterns. Sensor data can indicate the conditions present and the inputs to the model 112 at a given time, with subsequent sensor data showing what the intended or desired movement actually was, even if it was not what the model predicted or what the exosuit 102 attempted. By interpreting the data collected, the system 100 can infer what the correct commands would have been to support the movement of the user, and this can be used to further train the model 112 for an individual's own behavior and idiosyncratic movement style.

There are many other ways that the system 100 can adjust the control scheme for the exosuit 102 for the user in addition to or instead of adjusting the training state of the models 112. One is to adjust the selection of which models 112 to use in which situation. A library of various models 112 can be available, with different models used for different movement types (e.g., standing, sitting, walking, etc.), or for different gait types, or different ages. The tracked data about a user can adjust which model is selected and when the model transitions occur. For example, there may be three different models for generating “standing up” control instructions, and a first of these three may be initially used for a user. The tracked data for the user, however, may show that the actuation of the exosuit 102 using this model lags the user movement to stand. As a result, the exosuit 102 can be configured to use a second of the “standing up” models, one that provides a faster standing movement that better fits the sensor data pattern detected for the user. The change can be made in various different ways, such as providing the new model to the exosuit 102 over a network, assigning the second model to be used by the exosuit 102, changing probability scores or weights that the exosuit 102 uses to select which model 112 to use, or updating training of a model that selects other models. As another way to change model results, the system 100 can adjust weights or multiplication factors that are applied to a model's output. For example, rather than adjust a model's training, the system may adjust another parameter that mulitplies some weighting factor to the model output (e.g., x1.2 to make a 20% increase, or 0.9 to cause a 10% decrease), etc.

As another example, a control scheme can be adjusted by changing the thresholds or other settings that are used to initiate action by the exosuit 102. For example, thresholds for timing and positions of joints can be set to distinguish between different modes of operation or different movements performed by the exosuit 102. These thresholds may be quite different for different users. From the tracked data, the system 100 can customize the thresholds to align with a user's pattern. For example, a default threshold may detect that a sitting motion may be beginning when leg flexion decreases to one hundred and ten degrees. However, tracked data may show that the particular user has a wider range of movement for non-sitting movements, so sitting is more reliably detected after leg flexion decreases to one hundred degrees. In general, many different parameters can be changed, including dialing up or down assistance generally; applying offsets, scaling factors, or other adjustments to control signals output by a model 112; creating or adjusting context-dependent rules for how the exosuit 102 behaves for a specific user; etc.

When training models 112 or adjusting other parameters, the system 100 can use various tracked parameters in the optimization of the control scheme. For example, the system 100 can have a target amount of assistance that the exosuit 102 provides, and the system can adjust the models 112 and other parameters to better provide that level of assistance. Similarly, goals or targets can be set for the level of stability a user achieves with the exosuit 102 or for gait parameters (e.g., stride length, stride duration, when gait peaks), and the system 100 can tune the models 112 and other assistance parameters toward achieving those values. In some cases, the targets and changes are incrementally adjusted based on a user's own history or baseline. In other words, based on an observed gait, the system 100 can gradually and repeatedly adjust the control scheme over time so that the assistance provided by the exosuit 102 helps move toward improved gait patterns (e.g., gradually increasing stride length, gradually weaning the user to a lower level of assistance, etc.).

In some cases, the exosuit 102 can be used in a training session or enrollment phase where the user performs predetermined types of movements. The sensor data gathered about the user's behavior for these known movements can be used to make adjustments to the control scheme. For example, the user can be asked to perform enrollment or training actions with the exosuit 102, with the user performing predetermined types of movements with the exosuit. The exosuit 102 or a user device can instruct the use to take five steps, to sit down and then stand up, or perform other movements. The system 100 then characterizes the movement patterns, timing, and sensor signals that occur during these known movements and enters them in a user profile for the user. The system 100 then uses the user profile to select parameters that are used to provide assistance. This can include selecting, based on the user's movement patterns, which models 112 to use for different types of movements, and which triggers or thresholds to use for transitions between models 112 and movements. The data can also be used to adjust or train parameters of the models themselves.

In more detail, the exosuit 102 can be used in a training session with the user using the exosuit 102 with the user's typical gait and daily usage situations. The user can move the exosuit 102 through typical movements like walking, sitting, and climbing stairs. Often different machine learning models are used for these different activities, although generalized models can also be used which can handle ranges of speeds, inclines, and other situations. During the training session, the system takes into account characteristics of the user (e.g., height, age, medical condition) and the types of activities the user will perform can be known in advance. The system 100 can learn from the physiological parameters and results of sensing which data signals different situations or attempted user movements. As an example, the system 110 can learn that if heart rate is decreasing and the user is transitioning to a new activity, then a particular model should be used. In general, the tracked information can be used to fine tune the strategy for selecting the appropriate activity model 112 used to control the exosuit 102. Over the course of training and general use, some model predictions are a better match to the user's movement than others. The system 100 can measure the user actions and responses of the exosuit 102 with certain models, and the system 100 can detect when a model 112 provided an incorrect response, e.g., instructed actuation that was too strong, not strong enough, too late, too early, etc. From these responses, the system can modify the model output through training of the models 112 or can change the parameters used for switching or selecting between models to use at different times.

As discussed above, unsupervised training can also be used to select models and other parameters. The system 100 can collect a user's sensor data (e.g., gait data, movement patterns, level of assistance provided, etc.). The system 100 can then classify the sensor data or assign it to one of various clusters, based on the similarity of the user's sensor data with that of other users. Based on the clustering or classification, the system 100 can then select a model 112 that worked best for others whose sensor data had the same classification or clustering, indicating that those people that had similar gait or assistance needs. In some cases, the system 100 can cluster and group stored sensor data for various individuals. The patterns or data sets can be grouped by similarities in gait patterns, or based on other factors, such as lifestyle, age, height, medical condition, etc. From these, the system 110 builds a library or vocabulary of sensor data patterns with corresponding assistance parameters (e.g., thresholds, settings, models 112, etc.). For an individual, the system 100 can cluster the particular user's sensor data into one or more of the clusters based on the similarity of the user's sensor data patterns with those in the clusters. Regardless of how the clusters are originally defined, the similarity analysis can help select the correct assistance parameters for a user. For example, there may be models 112 defined for different age groups. Regardless of a particular user's actual age, the system 100 can match the specific sensor data patterns of the user with the reference patterns for a cluster, and retrieve the model that best fits. In other words, if the data observed looks like a pattern of some in a particular age group or with a particular heath condition, the system 100 can use that similar “style” of walking to select the models 112 used. In some cases, the system can get a time series of sensor data for a user, and then compare the time series with those of other data sets, to determine the best model 112 for that person and the activity to be performed. The model may be adapted for the user through further training based on the user's data patterns or other similar data patterns or others. For this process, no training labels need to be set, the system 100 can group walking patterns on how they appear in the data set.

FIG. 2 depicts an example of an environment 200 in which a device 202 receives, for presentation, personalized metrics or a recommendation from a personalized metrics system 214. The device 202 can present some of the received information in a user interface 204, using a speaker, e.g., when a computer assistant presents some of the received information audibly, or both.

For example, an exosuit, e.g., the exosuit 102 described with reference to FIG. 1, or a device connected to the exosuit, e.g., the user device 114, can provide personalized assistance metrics, e.g., for presentation on a display included in the exosuit or another device such as a smart watch. The personalized assistance metrics can provide a user with feedback about the exosuit, their use of an exosuit, or both. For instance, the personalized assistance metrics can indicate an amount of assistance a user who was wearing an exosuit received from the exosuit, e.g., for a predetermined period of time such as a day.

The personalized assistance metrics can indicate a change in an amount of assistance the exosuit provided. For example, the personalized assistance metrics can indicate that the user received a different amount of assistance, e.g., less, for a current time period, e.g., the past 24 hours, compared to a prior time period, e.g., yesterday or an average for the past week or a day last week.

The personalized assistance metrics can indicate a caloric measurement. For instance, a system can determine a first amount of caloric expenditure during a first period of time, e.g., two days ago, and a second amount of caloric expenditure during a second period of time, e.g., yesterday. The system can determine a difference between the first amount and the second amount that represents an amount of work that the person didn't do, an amount of energy the person saved, or both. The amount of work that the person didn't do or the amount of energy that the person saved can be a result of an exosuit providing assistance to an exosuit user. The amounts of energy can change over time as the exosuit system adapts to the exosuit user's patterns, e.g., of walking or otherwise using the exosuit.

In some examples, a system can determine the personalized metrics as a fraction of a total measurement for a day. For instance, the system can determine a difference between a first amount and a second amount. The system can use the difference to determine a fraction of the total measurement for a day, e.g., by dividing the difference by the total amount for the day. The total measurement for the day can be for the first period of time, the second period of time, or a combination, e.g., average, of the two time periods.

In some implementations, the personalized assistance metrics can indicate an amount of energy saved by an exosuit. For instance, a system can determine an amount of energy used by an exosuit for a first period of time and a second period of time. The system can determine a difference in the amounts of energy for the two time periods. The difference can indicate an amount of energy savings by the exosuit, e.g., as a result of an exosuit adapting to the exosuit user's patterns.

The personalized assistance metrics can indicate a physical attribute of an exosuit user. For example, the metrics can indicate that the user was 15% more stable, able to run 10% faster, or both, from one time period, e.g., last week, to another time period, e.g., this week. A system can determine the metrics using gait measurements, a number of steps taken, or other appropriate physical attribute data. Gait measurements can include length, duration, gait type, or when the gait peaks.

The metrics can indicate a change in a number of heart beats or a percentage of heart beats between two time periods. For instance, a user's heart rate can change as they exercise more, proceed in a physical therapy program, or both. A system can use the heart rate change to determine personalized assistance metrics, a recommendation on an action to change, or both. The personalized assistance metrics can indicate that “you saved 15,000 heartbeats because you used the exosuit.”

A system can determine metrics for a person based on their performance wearing an exosuit and not wearing an exosuit. The metrics can indicate a change in the performance under the two circumstances. For instance, the system can determine a metric that indicates an amount of energy saved by the person when the person wears an exosuit.

The personalized assistance metrics can indicate an index 206 that represents an amount of assistance a user who was wearing an exosuit received from the exosuit. The exosuit can calculate the index 206 based on a user's use of an exosuit. The exosuit can provide the index 206 for presentation to the user as an indicator about how the user is using the exosuit.

A system can determine the index using one or more personalized assistance metrics described here. For instance, a system can determine anonymous historical data for a group of exosuit users. The system can determine an index for an exosuit user by comparing historical data for the user with historical data for the group of exosuit users. The index can represent a measure of a person's historical data against a global standard, e.g., based on the group of exosuit users. For example, the anonymous historical data can indicate an average amount of energy saved. In this example, the index can indicate an amount of energy saved by the person compared to the average, e.g., “you saved five percent, since the global standard is three percent, you saved sixty percent more energy than the average person” with sixty percent as the index value.

The index can be any appropriate value, based on any appropriate type of historical data for exosuit use, that indicates whether an exosuit user is improving based on their exosuit use. In some implementations, the index can represent a score that does not have any units. For instance, a system can determine the score using historical data of different types for a user, such as a combination of heart rate, gait, and energy use. A system can use the score to track a user's performance, an exosuit's performance, or both, over time. The system can generate recommendations using the score, or present the score, to indicate a change in performance over time.

In some implementations, the index can include a pattern for an exosuit user compared to a global pattern. For instance, the system can determine a gait pattern for an exosuit user and receive data that indicates a global gait pattern for other exosuit users. The system can cause presentation of a user interface that indicates the user's gait pattern with respect to the global gait pattern. The index can indicate how close the user's gait pattern, or aspects of the user's gait pattern, is to the global gait pattern.

The index 206 can indicate changes the user should make in their use of the exosuit, an efficiency of the exosuit given the user's interaction with the exosuit, or some other data about the user's use of the exosuit. For example, a higher index 206 can indicate that the exosuit has a high efficiency, or has a high accuracy in predicting an action to perform. In some examples, a lower index 206 can indicate a low exosuit accuracy. The index 206 can indicate that the user is more likely to improve quickly, e.g., recover from a broken leg, when the index is high, e.g., and the exosuit has a high accuracy. The index 206 can indicate that the user should make adjustments to how they use the exosuit when the index is low, e.g., and the exosuit has a low accuracy.

In some implementations, the index 206 can represent a cooperation index. A higher score can indicate that the user is doing a good job and getting benefits from the exosuit. A lower score can indicate that the user is not getting as much benefit from the exosuit as they could. A higher index 206 can indicate that the system does not recommend many changes to the user's use of the exosuit. A lower index 206 can indicate that the system recommends more changes to the user's use of the exosuit. Data associated with the index, e.g., included in the user interface 204 near the index 206 value or presented upon receipt of data indicating selection of the index 206, can indicate recommended actions for the user to perform, e.g., to prompt the user to change their use of the exosuit.

In some examples, device 202 can present personalized assistance metrics that indicate an amount of energy a user who was wearing an exosuit saved by using the exosuit, an amount of additional work the user was able to perform, or both. For example, when a user who was or currently is wearing an exosuit was able to walk 500 extra steps based on assistance provided by the exosuit, the personalized assistance metrics could indicate this additional distance walked. In these examples, the device 202 can use the personalized assistance metrics to present a note to the user, e.g., “You got a lot of assistance today. This helped you take 500 steps more than yesterday.”

In some implementations, the device 202 can use the personalized metrics to determine, present information about, or both, trends in an exosuit's use. For instance, when the personalized metrics do not include trend information, the device 202 can analyze the personalized metrics to determine trends in the data over time. The device 202 can use data stored in a memory, e.g., historic personalized metrics, along with the personalized metrics received from the personalized metrics system 214 to determine the trends. The historic personalized metrics can be personalized metrics that the device 202 received from the personalized metrics system 214 previously, e.g., prior to the receipt of the current personalized metrics.

The trend information can indicate a difference between personalized metrics for a current time period compared to a prior time period. For instance, when the time period is the current day, the device 202 can present an activity user interface element 208 that indicates that the user was “active for 1 more hour today than yesterday.”

In some implementations, the device 202 can present, in the user interface 204, a trend graph 210 with trend information. The trend graph 210 can identify a number of hours a user was active each day, e.g., when the user was wearing an exosuit. For instance, the trend graph 210 can indicate that the user was active for about three hours on Tuesday, about four hours on Wednesday, about three and a half hours on Thursday, about three hours on Friday, about three and a half hours on Saturday, about for hours Sunday, and about five hours Monday.

The device 202 can present recommendation information about the types of activities performed by the user, in the user interface 204 or another user interface. The recommendation information can indicate whether individual activities are likely to improve the user's health or not likely to improve the user's health. For example, when a user has a broken leg and is using a leg exosuit, the device 202 can receive recommendation information from the personalized metrics system 214 that identifies activities performed by the user and whether those activities will help the user's leg to heal properly.

The recommendation information can be any appropriate type of feedback for a user. In some implementations, the recommendation information can be color coded, a thumbs up or down, a value that indicates a level of assistance provided by the exosuit, or any combination of two or more of these. The device 202 can provide the recommendation information to help motivate a user in correctly using an exosuit.

The device 202 receives the personalized metrics, or the recommendation, from the personalized metrics system 214 via a network 212. The personalized metrics system 214 analyzes exosuit sensor and control data 216 for an exosuit associated with the device 202. When the device 202 is an exosuit, the exosuit sensor and control data 216 is for the device 202. When the device 202 is separate from an exosuit, e.g., the device 202 is a smart watch, the exosuit sensor and control data 216 can be for an exosuit associated with the same user account as the device 202.

After the personalized metrics system 214 receives the exosuit sensor and control data 216, a historical data analysis engine 218 in the personalized metrics system 214 analyzes historical data stored in the exosuit sensor and control data 216. The historical data analysis engine 218 can be a computer or multiple computers included in the personalized metrics system 214. The historical data analysis engine 218 analyzes the historical data to determine trends in the data. Some examples of trends can include how much assistance an exosuit provided to a user wearing the exosuit, whether the user was properly using the exosuit, or improvements to the user's health over time, e.g., increased mobility, activity, or both.

The historical data analysis engine 218 can determine, using the sensor data, actions that a user repeatedly performs incorrectly. The actions can be performed incorrectly based on the user's use of an exosuit, a physical therapy program for the user, or both. For instance, when the user has a broken leg, the historical data analysis engine 218 can determine whether the user is standing up correctly, walking correctly, or should be moving in a certain way, e.g., running, given the user's progress in a physical therapy program. If the user is moving more quickly than they should be since the user is in the beginning stages of physical therapy, the historical data analysis engine 218 can determine, from the sensor data, that the user is moving more quickly than they should.

A recommendation engine 220 included in the personalized metrics system 214 receives data from the historical data analysis engine 218 about a user, an exosuit, or both. The recommendation engine 220 analyzes the received data to generate personalized metrics, a recommendation, or both. For example, when a user is moving more quickly than they should, the recommendation engine 220 can generate recommendation data that indicates that the user should be moving more slowly. The recommendation data can indicate a reason why the recommendation was generated, e.g., that the user is still in the early stages of a physical therapy program.

The personalized metrics system 214 is an example of a system implemented as computer programs on one or more computers in one or more locations, in which the systems, components, and techniques described in this document are implemented. In some examples, the personalized metrics system 214 is part of the same system as the training system 116 described with reference to FIG. 1.

The device 202 may include a personal computer, a mobile communication device, and other devices that can send and receive data over the network 212. The network 212, such as a local area network (LAN), wide area network (WAN), the Internet, or a combination thereof, connects the device 202, and the personalized metrics system 214 (in implementations when the personalized metrics system 214 is not part of the device 202). The personalized metrics system 214 may use a single server computer or multiple server computers operating in conjunction with one another, including, for example, a set of remote computers deployed as a cloud computing service.

The personalized metrics system 214 can include several different functional components, including the historical data analysis engine 218, and the recommendation engine. The various functional components of the personalized metrics system 214 may be installed on one or more computers as separate functional components or as different modules of a same functional component. For example, the historical data analysis engine 218, and the recommendation engine of the personalized metrics system 214 can include one or more computers in the system or be implemented as computer programs installed on one or more computers in one or more locations that are coupled to each through a network. In cloud-based systems for example, these components can be implemented by individual computing nodes of a distributed computing system.

Further to the descriptions above, a user may be provided with controls allowing the user to make an election as to both if and when systems, programs, or features described herein may enable collection of user information (e.g., information about a user's social network, social actions, or activities, profession, a user's preferences, or a user's current location), and if the user is sent content or communications from a server. In addition, certain data may be treated in one or more ways before it is stored or used, so that personally identifiable information is removed. For example, a user's identity may be treated so that no personally identifiable information can be determined for the user, or a user's geographic location may be generalized where location information is obtained (such as to a city, ZIP code, or state level), so that a particular location of a user cannot be determined. Thus, the user may have control over what information is collected about the user, how that information is used, and what information is provided to the user.

A number of implementations have been described. Nevertheless, it will be understood that various modifications may be made without departing from the spirit and scope of the disclosure. For example, various forms of the flows shown above may be used, with steps re-ordered, added, or removed.

Embodiments of the invention and all of the functional operations described in this specification can be implemented in digital electronic circuitry, or in computer software, firmware, or hardware, including the structures disclosed in this specification and their structural equivalents, or in combinations of one or more of them. Embodiments of the invention can be implemented as one or more computer program products, e.g., one or more modules of computer program instructions encoded on a computer readable medium for execution by, or to control the operation of, data processing apparatus. The computer readable medium can be a machine-readable storage device, a machine-readable storage substrate, a memory device, a composition of matter effecting a machine-readable propagated signal, or a combination of one or more of them. The term “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them. A propagated signal is an artificially generated signal, e.g., a machine-generated electrical, optical, or electromagnetic signal that is generated to encode information for transmission to suitable receiver apparatus.

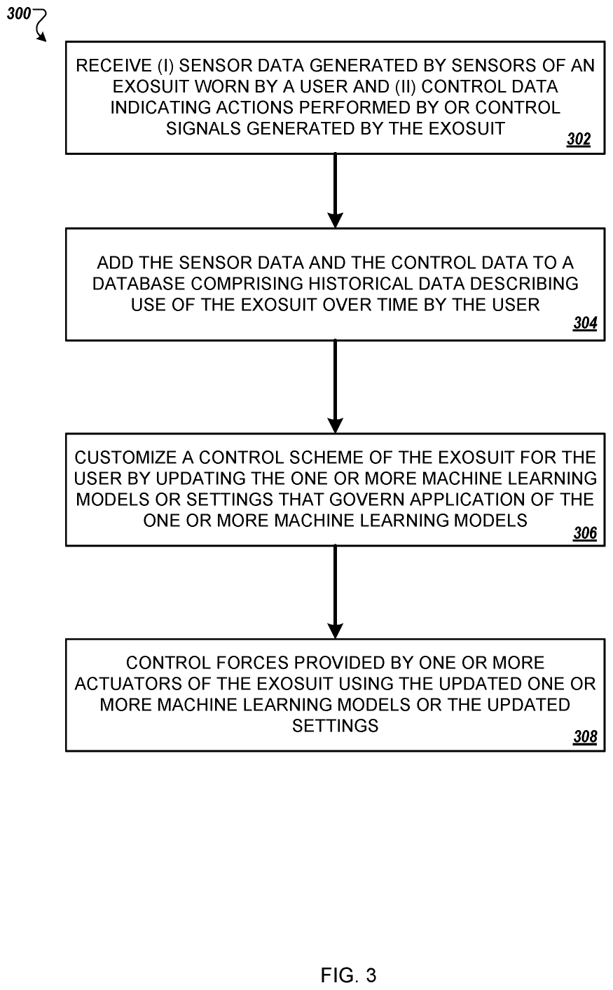

FIG. 3 is a flow chart illustrating an example process 300 for controlling an exosuit using sensor data collected over time. As an example, the process 300 can be performed by the exosuit 102 using the machine learning model 112.

The process 300 includes receiving (i) sensor data generated by sensors of an exosuit worn by a user and (ii) control data indicating actions performed by or control signals generated by the exosuit (302). With respect to FIG. 1, the sensor data may be generated by sensors of the exosuit 102 when the exosuit 102 is being worn by the user. For example, the exosuit 102 can generate the sensor data 108a when the user is wearing the exosuit 102. The control data may indicate actions performed by the exosuit 102 or the user when the user is wearing the exosuit 102, or control signals generated by the exosuit 102 when the user is wearing the exosuit 102. For example, the control data 108b can be generated using the sensor data 108a.

The control data may be determined using one or more machine learning models. For example, with respect to FIG. 1, the exosuit 102 can use the machine learning model 112 to generate the control data 108b. In generating the control data 108b, the exosuit 102 can provide the sensor data 108a to the machine learning model 112 as input for the machine learning model 112.

The process 300 includes adding the sensor data and the control data to a database comprising historical data describing use of the exosuit over time by the user (304). For example, the exosuit 102 can include a transceiver that it uses to communicate over a network, such as a cloud computing network. In response to obtaining the sensor data 108a collected over a collection or observation period, the exosuit 102 may transmit the sensor data 108a to the training system 116. In response to receiving the sensor data 108a, the training system 116 can proceed to update historic data for the user (“User A”) by storing the sensor data 108a in the database 120. The training system 116 may also use the sensor data 108a to update other databases, such as the database 118. For example, the sensor data 108a may be stored in a particular area of the general exosuit database 118 reserved for users with particular characteristics that the user wearing the exosuit 102 meets. For example, based on age and/or medical conditions of the user wearing the exosuit 102, the training system 116 can use the sensor data 108a to update a particular area of the database 118 reserved for users within a certain age range that the user meets and/or having certain medical conditions that the user has.

The process 300 includes customizing a control scheme of the exosuit for the user by updating the one or more machine learning models or settings that govern application of the one or more machine learning models (306). For example, with respect to FIG. 1, customizing a control scheme of the exosuit 102 for the user can be realized by updating the machine learning model 112 and proceeding to use the updated machine learning model 112 to generate the control data 108b. There are various ways that the exosuit 102 can perform the update. As an example, the exosuit 102 can update the machine learning model 112 using newly acquired sensor data obtained while the user is wearing the exosuit 102 and/or performing a certain set of actions while wearing the exosuit 102. In more detail, the user may receive instructions through the user device 114 to perform one or more particular actions while wearing the exosuit 102, such as walking a particular distance, standing up, jogging, running, walking up stairs, or walking down stairs. The exosuit 102 may track the sensor data generated from onboard sensors while the user is performing these predetermined actions and proceed to use this sensor data obtained over the collection period defined by when the user was performing the actions to update the machine learning model 112.

Prior to customization of the control scheme, the machine learning model 112 may be a base model. For example, the machine learning model 112 may be a base model for all users or for all users having certain characteristics (e.g., age, medical conditions, height, weight, etc.). After collecting the sensor data 108a, the exosuit 102 or the training system 116 can proceed to use the sensor data 108a to update the machine learning model 112. The updated machine learning model 112 may then be used to customize the control scheme by generating the control data 108b that is personalized for the user. As an example, the sensor data 108a that the user struggled or failed to stand up while wearing the exosuit 102 after attempting to stand up. The exosuit 102 may train the machine learning model 112 using the current actuator settings, the observed effect (e.g., failure to stand), and the desired effect (e.g., successful standing). After updating the machine learning model 112, the exosuit 102 may later provide as input to the machine learning model an indication of the action to be performed by the user (e.g., standing action). The resulting output of the updated machine learning model 112 may include control settings for the actuators that are different with respect to the control data 108b such that at least a subset of the actuators are instructed to provide more force during the standing action. The sensor data 108a may also indicate other information the exosuit 102 or the training system 116 can use to update the machine learning model 112. For example, the sensor data 108a may indicate particular movements and/or movement behaviors for the user that are used to train the machine learning model 112. The resulting control scheme generated using the updated machine learning model 112 may provide for countering these movements and/or movement behaviors, particularly if they are undesirable or dangerous (e.g., deviate a particular percent threshold from predefined/ideal movement(s) and/or from typical movements observed from the user or other users).

In some implementations, the exosuit 102 or the training system 116 may also use the obtained sensor data or data obtained from other sensors (e.g., accelerometer data from the user device 114) to determine or verify which actions the user is performing or has performed. The exosuit 102 or training system 116 can use this information to categorize subsets of the sensor data 108a such that the subsets correspond to particular actions. For example, using accelerometer data of the user device 114, the training system 116 can determine times when the user is performing a standing action. After receiving the sensor data 108a, the training system 116 can extract a subset of the sensor data 108a that corresponds to the times when the user was determined to be performing the standing action. The training system 116 may then use this subset to update a particular portion of the user's historical data in the database 120, specifically a portion corresponding to standing actions for the user. The training system 116 can also use this subset of the sensor data 108a to update a particular portion of the database 118 corresponding to standing actions for all users in general or for users with particular characteristics.

Another technique that the exosuit 102 can use to update the machine learning model 112 is to update the machine learning using historical data for the user. As an example, after obtaining sensor data collected over a collection period and corresponding to particular actions, the exosuit 102 or the training system 116 can use the sensor data 108a to update the historical data for the user and proceed to use the updated historical data to update the machine learning model 112. Additionally or alternatively, the exosuit 102 or the training system 116 can use the sensor data 108a to update a portion of the general exosuit data in the database 118.

In some implementations, the training system 116 updates the one or more machine learning models. For example, in accordance with the techniques described above, the training system 116 may update one or more machine learning models using the sensor data 108a, using data obtained from the database 120 (e.g., after the database 120 has been updated using the sensor data 108a), and/or using data obtained from the database 118 (e.g., after the database 118 has been updated using the sensor data 108a).

In some implementations, the exosuit 102 or the training system 116 instructs the user to perform particular actions periodically or in response to certain events. For example, the training system 116 may provide instructions to the user device 114 every month that provide for the user to perform the same, modified, or different set of actions. The sensor data collected during these periodic sessions can be used to periodically update the machine learning model 112, to select a different machine learning model for generating the control scheme, and/or for changing settings for selecting the machine learning model 112 or a different machine learning model (e.g., parameters for selecting the machine learning model 112 may be changed based on the sensor data such that machine learning model 112 is only selected for the user when they are performing a jogging actions, for users that are below a certain age, for users that are above a certain age, for a particular age range of users, for users having a certain medical condition, etc.).

As another example, the training system 116 may provide instructions to the user device 114 in response to detecting certain events. In more detail, if sensor data indicates that the user has fallen while wearing the exosuit 102, the training system 116 may send instructions to the user devices 114 informing the user to perform certain actions as the event may indicate that (i) a different machine learning model should be selected, (ii) that settings for selecting a particular machine learning model should be changed, and/or (iii) that the machine learning model 112 should be updated.

In some implementations, in customizing the control scheme, a different machine learning model is selected for the user. For example, with respect to FIG. 1, based on the sensor data 108a, the exosuit 102 or the training system 116 may select a machine learning model other than the machine learning model 112 to use for the user and/or for the particular action the user is performing or attempting to perform. For example, the sensor data 108a may be obtained using an initial control scheme generated using the machine learning model 112 (e.g., which may be initially selected based on characteristics of the user such as height, sex, weight, and age).

The sensor data 108a may indicate, however, that results of using this control scheme were sufficiently undesirable such that a different machine learning model should be selected. For example, the sensor data 108a may indicate that one or more undesirable movements were observed (e.g., movement typically associated with injuries) and/or that one or more attempted actions were not successful (e.g., user failed to stand up from a sitting position). The exosuit 102 or the training system 116 may proceed to use the sensor data 108a to, for example, place the user into a different group of users (e.g., even if the user's characteristics don't match one, multiple, or all of the characteristics typically associated with that group) that correspond to a different machine learning models. For example, the exosuit 102 or the training system 116 may use the sensor data 108a and one or more clustering algorithms to identify the nearest cluster for the user. Each cluster may be associated with a machine learning model that has been historically shown to produce the best control scheme for users in the respective cluster (e.g., movements closest to ideal movements or to average movements observed in a healthy set of persons, least amount of injuries observed overtime, best feedback from users, etc.). After identifying this cluster, the exosuit 102 or training system 116 can look up the control scheme machine learning model(s) associated with the cluster and proceed to use those machine learning model(s) to generate the next control scheme for the user (e.g., generate the control data 108b).

In some implementations, the exosuit 102 or the training system 116 performs the clustering using sensor data that corresponds to particular actions. For example, the sensor data may correspond to data collected when the user is performing predetermined types of movements while wearing the exosuit 102.

In some implementations, the exosuit 102 or the training system 116 performs the clustering using sensor data that does not correspond to particular actions. For example, the sensor data may correspond to data collected over a predetermined amount of time (e.g., one day, one week, one month, etc.) while the user wearing the exosuit 102. The exosuit 102 or the training system 116 may proceed to provide this unsupervised data as input to one or more clustering algorithms to identify a group of other users that the user moves most similarly too (e.g., user has a walking gait that is most similar to users in this group, user has undesirable movements most similar to users in this group such as significant lateral motion while standing up) and/or to identify the machine learning model that will produce the most desirable control scheme for the user (e.g., least injuries, movements closest to ideal movements, movements closest to typical movements of health individuals, etc.).

In some implementations, in customizing the control scheme, the settings or parameters for a machine learning model are modified. These settings may be modified for the user particularly, e.g., based on historical data for the user. Alternatively, these settings may be modified for all users or all users in a particular group (e.g., corresponding to a particular cluster). As an example, certain machine learning models may be triggered by the detection of particular sensor output levels. For example, the machine learning model 112 may be selected for generating the control scheme when an accelerometer on the exosuit 102 outputs a reading of 0.5 G which may correspond to a user attempting to stand up. However, if the sensor data 108a or feedback from the user indicates that this was not the action that user was trying to perform, then the settings for selecting the machine learning model 112 may be changed to, for example, 0.6 G. Alternatively, if the control scheme produced by the machine learning model 112 was not successful in assisting the user to perform the standing action (or did not sufficiently assist the user with the desired action), the settings for the machine learning model 112 may be changed such that a different machine learning model is selected when the accelerometer outputs a reading of 0.5 G corresponding to the standing action.

The process 300 includes controlling forces provided by one or more actuators of the exosuit using the updated one or more machine learning models or the updated settings (308). For example, when the machine learning model 112 has parameters (e.g., neural network weights, classifier parameters, or other parameters) change through further training based on the user's tracked data, then the updated machine learning model 112 can be used to produce the instructions to control the actuators of the exosuit 102. In other cases, the algorithm or model used to select between different machine learning models 112 is updated for the user, and so the updated algorithm or model is used to select which model 112 to use to generate the control instructions. Other thresholds or parameters that are customized for the user can also be used.

As an example, based on the customized control scheme, the exosuit 102 can look up particular settings or actions for each of the actuators in the exosuit 102. The settings or actions may call for the application of a particular force or the application of a particular force pattern over time. For example, the control scheme can provide that a first pneumatic actuator of the exosuit 102 be engaged at a force of 9.8 N for a time of three seconds (e.g., to assist the user with a standing function) and, thereafter, be engaged with a force of 2.4 N. Instead of being based on preset time, the force pattern may be based on particular events. For example, the control scheme can provide that the first pneumatic actuator of the exosuit 102 be engaged at a force of 1.2 N until sensor data indicates that the user is attempting to stand up, at which point the force pattern of the control scheme can provide for increasing the force output of the first pneumatic actuator to 9.8 N. Once second sensor data indicates that the user has completed the standing action, the force pattern of the control scheme may provide for lowering the force output of the first pneumatic actuator to 2.4 N. Of course, the timing, force, and other parameters of actuation can be determined from outputs of machine learning models 112 provided in response to input to the models 112 that is based on sensor data indicating the current context and recent sensor measurements of the exosuit 102.

A computer program (also known as a program, software, software application, script, or code) can be written in any form of programming language, including compiled or interpreted languages, and it can be deployed in any form, including as a standalone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a tablet computer, a mobile telephone, a personal digital assistant (PDA), a mobile audio player, a Global Positioning System (GPS) receiver, to name just a few. Computer readable media suitable for storing computer program instructions and data include all forms of non volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, embodiments of the invention can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

Embodiments of the invention can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the invention, or any combination of one or more such back end, middleware, or front end components. The components of the system can be interconnected by any form or medium of digital data communication, e.g., a communication network. Examples of communication networks include a local area network (“LAN”) and a wide area network (“WAN”), e.g., the Internet.

The computing system can include clients and servers. A client and server are generally remote from each other and typically interact through a communication network. The relationship of client and server arises by virtue of computer programs running on the respective computers and having a client-server relationship to each other.

While this specification contains many specifics, these should not be construed as limitations on the scope of the invention or of what may be claimed, but rather as descriptions of features specific to particular embodiments of the invention. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

In each instance where an HTML file is mentioned, other file types or formats may be substituted. For instance, an HTML file may be replaced by an XML, JSON, plain text, or other types of files. Moreover, where a table or hash table is mentioned, other data structures (such as spreadsheets, relational databases, or structured files) may be used.

Particular embodiments of the invention have been described. Other embodiments are within the scope of the following claims. For example, the steps recited in the claims, described in the specification, or depicted in the figures can be performed in a different order and still achieve desirable results.

What is claimed is:

1. A computer-implemented method comprising:
receiving, by one or more computing devices, (i) sensor data generated by sensors of an exosuit worn by a user and (ii) control data indicating actions performed by or control signals generated by the exosuit based on the sensor data while worn by the user, wherein the control data is determined using one or more machine learning models;
adding, by the one or more computing devices, the sensor data and the control data to a database comprising historical data describing use of the exosuit over time by the user;
customizing, by the one or more computing devices, a control scheme of the exosuit for the user by updating the one or more machine learning models or settings that govern application of the one or more machine learning models, wherein the control scheme is customized using the historical data for the user; and
controlling, by the one or more computing devices, forces provided by one or more actuators of the exosuit using the updated one or more machine learning models or the updated settings.

2. The method of claim 1, comprising:
receiving data that identifies user input that indicates whether an action by the exosuit should have been performed; and
adding the data that identifies the user input to the database, wherein updating the one or more machine learning model comprises updating the one or more machine learning models using the historical data and the user input.

3. The method of claim 1, wherein:
the one or more machine learning models comprises a generic model; and
updating the one or more machine learning models for the user using the historical data comprises updating the generic model using a transfer learning process and the sensor data received by the exosuit during use of the exosuit by the user.

4. The method of claim 1, wherein updating the one or more machine learning models using the historical data comprises updating the one or more machine learning models using an unsupervised learning process and movement patterns of the user identified from the historical data.

5. The method of claim 1, wherein updating the one or more machine learning models using the historical data comprises updating the one or more machine learning models using a reinforcement learning process and data that identifies user input received from the user who wore the exosuit, the data indicating an assistance measure for the exosuit.

6. The method of claim 1, wherein:
the database includes second sensor data captured by sensors included in a plurality of other exosuits respectively worn by other users and second control data indicating actions performed by or control signals respectively generated by the other exosuits while worn by the corresponding other users; and
updating the one or more machine learning models comprises updating the one or more machine learning models using the sensor data, the control data, the second sensor data and the second control data that are included in the database of the historical data to obtain the updated one or more machine learning models.

7. The method of claim 6, comprising:
determining, using some of the sensor data or some of the control data from the database, an exosuit activity type; and
selecting, using the exosuit activity type, a particular machine learning model from a plurality of machine learning models for use by the exosuit when the user is wearing the exosuit.

8. The method of claim 7, wherein the exosuit activity type comprises one of: lifting an object, walking, sitting, standing, running, walking up stairs, or writing.

9. A non-transitory computer storage medium encoded with instructions that, when executed by one or more computers, cause the one or more computers to perform operations comprising:
receiving, by the one or more computers, (i) sensor data generated by sensors of an exosuit worn by a user and (ii) control data indicating actions performed by or control signals generated by the exosuit based on the sensor data while worn by the user, wherein the control data is determined using one or more machine learning models;
adding, by the one or more computers, the sensor data and the control data to a database comprising historical data describing use of the exosuit over time by the user;
customizing, by the one or more computers, a control scheme of the exosuit for the user by updating the one or more machine learning models or settings that govern application of the one or more machine learning models, wherein the control scheme is customized using the historical data for the user; and
controlling, by the one or more computers, forces provided by one or more actuators of the exosuit using the updated one or more machine learning models or the updated settings.

10. The non-transitory computer storage medium of claim 9, comprising:
receiving data that identifies user input that indicates whether an action by the exosuit should have been performed; and
adding the data that identifies the user input to the database, wherein updating the one or more machine learning model comprises updating the one or more machine learning models using the historical data and the user input.

11. The non-transitory computer storage medium of claim 9, wherein:
the one or more machine learning models comprises a generic model; and
updating the one or more machine learning models for the user using the historical data comprises updating the generic model using a transfer learning process and the sensor data received by the exosuit during use of the exosuit by the user.

12. The non-transitory computer storage medium of claim 9, wherein updating the one or more machine learning models using the historical data comprises updating the one or more machine learning models using an unsupervised learning process and movement patterns of the user identified from the historical data.

13. The non-transitory computer storage medium of claim 9, wherein updating the one or more machine learning models using the historical data comprises updating the one or more machine learning models using a reinforcement learning process and data that identifies user input received from a user who wore the exosuit, the data indicating an assistance measure for the exosuit.

14. The non-transitory computer storage medium of claim 9, wherein:
the database includes second sensor data captured by sensors included in a plurality of other exosuits respectively worn by other users and second control data indicating actions performed by or control signals respectively generated by the other exosuits while worn by the corresponding other users; and
updating the one or more machine learning models comprises updating the one or more machine learning models using the sensor data, the control data, the second sensor data and the second control data that are included in the database of the historical data to obtain the updated one or more machine learning models.

15. A system comprising:
one or more computers; and
one or more storage devices on which are stored instructions that are operable, when executed by the one or more computers, to cause the one or more computers to perform operations comprising:
receiving, by the one or more computers, (i) sensor data generated by sensors of an exosuit worn by a user and (ii) control data indicating actions performed by or control signals generated by the exosuit based on the sensor data while worn by the user, wherein the control data is determined using one or more machine learning models;
adding, by the one or more computing computers, the sensor data and the control data to a database comprising historical data describing use of the exosuit over time by the user;
customizing, by the one or more computers, a control scheme of the exosuit for the user by updating the one or more machine learning models or settings that govern application of the one or more machine learning models, wherein the control scheme is customized using the historical data for the user; and
controlling, by the one or more computers, forces provided by one or more actuators of the exosuit using the updated one or more machine learning models or the updated settings.

16. The system of claim 15, comprising:
receiving data that identifies user input that indicates whether an action by the exosuit should have been performed; and
adding the data that identifies the user input to the database, wherein updating the one or more machine learning model comprises updating the one or more machine learning models using the historical data and the user input.

17. The system of claim 15, wherein:
the one or more machine learning models comprises a generic model; and
updating the one or more machine learning models for the user using the historical data comprises updating the generic model using a transfer learning process and the sensor data received by the exosuit during use of the exosuit by the user.

18. The system of claim 15, wherein updating the one or more machine learning models using the historical data comprises updating the one or more machine learning models using an unsupervised learning process and movement patterns of the user identified from the historical data.

19. The system of claim 15, wherein updating the one or more machine learning models using the historical data comprises updating the one or more machine learning models using a reinforcement learning process and data that identifies user input received from a user who wore the exosuit, the data indicating an assistance measure for the exosuit.

20. The system of claim 15, wherein:
the database includes second sensor data captured by sensors included in a plurality of other exosuits respectively worn by other users and second control data indicating actions performed by or control signals respectively generated by the other exosuits while worn by the corresponding other users; and
updating the one or more machine learning models comprises updating the one or more machine learning models using the sensor data, the control data, the second sensor data and the second control data that are included in the database of the historical data to obtain the updated one or more machine learning models.
